(19) United States
(12) Patent Application Publication
(10) Pub. No.: US 2026/0284284 A1
(43) Pub. Date: Sep. 24, 2026

(54) DIALYSIS SYSTEM FOR TREATING SEPSIS

(71) Applicants: MEXBRAIN, Villeurbanne (FR); UNIVERSITE CLAUDE BERNARD LYON 1, Villeurbanne (FR); CENTRE NATIONAL DE LA RECHERCHE SCIENTIFIQUE - CNRS -, Paris (FR); INSERM (INSTITUT NATIONAL DE LA SANTÉ ET DE LA RECHERCHE MÉDICALE), Paris (FR); INSTITUT GUSTAVE ROUSSY, Villejuif (FR); VETAGRO-SUP INSTITUT D'ENSEIGNEMENT SUPÉRIEUR ET DE RECHERCHE, Marcy l'Etoile (FR)

(72) Inventors: Marco NATUZZI, Villeurbanne (FR); Thomas BRICHART, Lyon (FR); Fabien ROSSETTI, Villeurbanne (FR); Matteo MARTINI, Villars les Dombes (FR); François LUX, Lyon (FR); Olivier TILLEMENT, Fontaines Saint-Martin (FR); Jean-Luc PERFETTINI, Meaux (FR); Vanessa LOUZIER, Ecully (FR); Jeanne-Marie BONNET-GARIN, Lyon (FR); Jean-Yves AYOUB, Lyon (FR)

(21) Appl. No.: 19/280,936

(22) Filed: Jul. 25, 2025

Related U.S. Application Data
(63) Continuation of application No. 17/997,763, filed on Apr. 30, 2021, now abandoned.

(30) Foreign Application Priority Data
May 4, 2020 (FR) FR2004406

(51) Int. Cl. A61M 1/16

(52) U.S. Cl. CPC A61M 1/1621; A61M 1/1654

(57) ABSTRACT

Oxidative stress in a patient is reduced using a dialysis system. The dialysis system has a porous membrane and a vessel containing dialysis fluid with an additive therein that is polymeric and which has a size larger than the cutoff threshold of the membrane. In addition the additive contains at least one ligand specific for metal cations that circulated in the blood. The dialysis system can be used for treating sepsis caused by bacterial, parasitic, fungal or viral infections.

### CROSS-REFERENCE TO RELATED APPLICATIONS

This application is a continuation of application U.S. Ser. No. 17/997,763 filed on Nov. 2, 2022, which itself was a national stage filing under 35 USC 371 from International Application PCT/FR2021/050750 filed on Apr. 30, 2021, which itself claimed priority to FR2004406 filed on May 4, 2020.

### CONTEXT

Severe acute respiratory syndrome coronavirus 2 (SARS-COV-2) appeared for the first time in Wuhan, in China, at the end of the year 2019. Even though the majority of patients have a relatively good prognosis, COVID-19 entails significant mortality, for example close to 3.7% in certain studies¹. For seriously ill patients and those who died, in general no serious sign is observed at the start of the disease (only a slight fever, a cough or muscle pains).

However, the condition of patients deteriorates rapidly at an advanced stage of the disease. Various syndromes are associated with seriousness of the disease: (i) acute respiratory distress syndrome (ARDS), (ii) multiple organ failure, (iii) sepsis and septic shock^{2,3}.

In the recent past, coronaviruses have caused other respiratory diseases, namely severe acute respiratory syndrome (SARS) and Middle East respiratory syndrome (English acronym “MERS”)⁴.

The clinical signs, in particular fever, non-productive cough, dyspnea, myalgia, fatigue, radiography traces of pneumonia, etc . . . , are similar to the symptoms of SARS and of MERS.

ARDS is the immunopathological event common to COVID-19, SARS and MERS, and the main cause of death in COVID-19⁵.

ARDS most often occurs in elderly persons and those suffering from immunological disorders and comorbidities. The pneumonia syndrome can also be associated with a bacterial co-infection in serious cases and with sepsis⁶.

Cytokine shock, also called “cytokine storm” (in English “cytokine storm”), is considered to be one of the main causes of ARDS and multiple organ failure, and plays an important part in the process of worsening of the disease.

The cytokine storm is the uncontrolled and fatal systemic inflammatory response resulting from the release of large quantities of pro-inflammatory cytokines (for example IFN-α, IFN-γ, IL-1β, IL-6, IL-12, IL-18, IL-33, TNF-α and TGFβ) and chemokines (for example CCL2, CCL3, CCL5, CXCL8, CXCL9 and CXCL10).

Just as with MERS and SARS, persons suffering from COVID-19 can display high levels of IL-6, IFN-α and IFN-γ and of CCL5, CXCL8 and CXCL10 in the serum compared to those suffering from a mild/moderate disease^{7,8}.

The clinical predictors of the mortality of COVID-19 were determined from the study of 150 cases in Wuhan, in China. These predictors are elevated ferritin (1297.6 ng/ml on average in non-survivors against 614.0 ng/ml in survivors (p<0.001)) and an elevated level of IL-6 (p<0.0001), thus suggesting that the mortality is due to hyper-inflammation of viral origin⁹.

These results have been conformed by a meta-analysis showing higher levels of IL-6 (4.6 pg/mL against 1.7 pg/mL) and serum ferritin (760.2 ng/ml against 408.3 ng/ml) for survivors versus non-survivors of COVID-19¹⁰. These data and the elevated level of IL-10 observed in the serious form of the disease are predictors of cytokine storm syndrome and underline the role of the inflammatory cytokines and iron in the development of serious cases of the disease. The authors thus suggest the use of the IL-6 and ferritin levels as predictors for monitoring the prognosis of the disease in patients suffering from COVID-19.

Blood purification therapy has already shown its potential for eliminating pathogenic antibodies or cytokines in many disease conditions and may be a valuable therapeutic means for COVID-19 patients¹¹.

Sepsis is currently defined as “an organ dysfunction secondary to deregulation of the response of the host to infection (bacterial, viral, fungal or parasitic) and threatening the vital prognosis” (Sepsis-3 Consensus Conference, 2016). As for septic shock, this is defined as “a sub-group of sepsis characterized by intense circulatory, metabolic and cellular abnormalities associated with a higher mortality than in sepsis”. Emphasis is placed on organ dysfunction. The complex, associated physiopathology, in response to the microbial invasion: inflammation and immunosuppression, a procoagulatory state, endothelial dysfunction and tissue hypoxia: the combination of these mechanisms leading in the end to organ failure.

The standard therapy in early management consists in initiating broad spectrum antibiotic therapy, identifying the causative agent(s), monitoring the hemodynamic parameters of the patients, installing pulmonary ventilation and administrating replacement fluids and vasopressor medication with the aim of maintaining a satisfactory mean arterial pressure (MAP). In spite of the institution of these aggressive and costly therapeutic measures, the mortality connected with short-term and long-term septic shock remains significant and the incidence of the disease constantly increasing. The prognosis is particularly bad. The mortality due to sepsis is estimated at 25.8% in intensive care units and 35.3% in hospitals¹².

In the world, it is estimated that 20 to 30 million people suffer from sepsis each year, and that almost 24,000 people die of it every day. The seriousness of sepsis is due in particular to an activation cascade leading to auto-amplified production of cytokines (the cytokine storm) which are relatively small proteins (less than 40 kDa)¹³.

On account of the central role of cytokine production during sepsis and the subsequent damage caused to the organs, blood purification has been proposed for treating sepsis, mainly using the so-called high volume hemofiltration technology and/or the so-called adsorbent technology¹⁴.

Adsorbent technology essentially consists in passing the blood through an adsorbent material, for example specific membranes or polymer beads, in order to retain the molecules produced in excess. A known example is in particular the commercial device CytoSorb©. These systems, although making it possible to extract molecules from the blood, are not specific to one type of molecule in particular.

Likewise in high volume hemofiltration, the majority of experimental and clinical studies reporting beneficial effects in inflammatory shock cases have been performed with very high filtration flow rates, often greater than 100 ml·kg⁻¹·h⁻¹ resulting in a major loss of plasma for the patient replaced by a substitution liquid reinjected into the extrarenal purification circuit. While it is relatively easy to compensate for the losses of water, salt and some simple chemical components with the replacement liquid of appropriate electrolyte composition, it is very difficult to compensate for all the essential components also lost during the high volume hemofiltration session.

Various mechanisms have been proposed to demonstrate the advantages of reduction of the level of cytokines in the blood: (i) the maximal concentration theory postulating that elimination of the cytokine peak during the early phase could stop the inflammatory cascade¹⁵, (ii) the cytokinetic theory suggesting that elimination of cytokines from the blood can create a cytokine gradient between the bloodstream and the tissues and thus lead to the elimination of the cytokines from the tissues owing to the equilibrium of the concentration of cytokines between the tissues and the bloodstream.

However, the main disadvantage of the present blood purification methods is the non-specific elimination of certain molecules, for example small blood proteins such as other regulatory anti-inflammatory cytokines. At present, none of the purification methods by use of an adsorbent on beads, porous supports or on membranes is sufficiently specific to one type of molecule. Moreover, clinical trials with the adsorbent technology are often few or inconclusive.

Particular devices making it possible to extract circulating molecules from the blood have in the course of time been developed.

The document WO 96/16666 relates to a method for the specific adsorption of pathogenic factors the presence whereof is increased during an HIV infection and which is correlated with a stage of acquired immunodeficiency.

Oxidative stress is a situation harmful to the body which causes biological damage. It occurs when the quantity of pro-oxidant molecules is greater than that of the anti-oxidants. The pro-oxidant molecules are mainly constituted of reactive oxygen species (English acronym “ROS”) and reactive nitrogen species (English acronym “RNS”). Under the conditions of sepsis, overproduction of ROS and of RNS is observed simultaneously in the blood circulation and in the damaged organs¹⁶.

It has long been known that the production of ROS is catalyzed by free iron in the Fenton and Haber-Weis reaction¹⁷. It has also been proved that copper is involved in the production of ROS by a reaction of the Fenton type¹⁸. An increase in free or weakly bound copper or in free iron (or redox active transition metals) is directly associated with a major increase in the production of ROS and in the damage which results therefrom.

An increase in free iron in the serum has recently been linked to an increase in mortality in patients suffering from sepsis in a retrospective study relating to 1891 patients¹⁹. Thus, a more elevated iron quartile has been linked to a significant increase in the mortality risk at 90 days. Still more significantly, an increase in the risk of death as a function of the quantity of iron has been seen when the iron level increases. This study is in agreement with others which link sepsis to elevated levels of various metals in patients²⁰. In this study, Cr (p<0.001), Fe (p=0.004), Ni (p=0.001), Cu (p<0.001) and Cd (p<0.0001) are significantly higher in the serum of the group suffering from sepsis than in that of the control group.

Owing to its importance for the growth of bacteria and its involvement in the production of ROS, therapeutic iron restriction has already been proposed as a strategy for treating sepsis and septic shock²¹. However, in spite of a certain success, the chelation of iron presents certain limits, in particular significant secondary effects, such as for example damage to the kidneys or exacerbation of infections for deferoxamine (DFO) which can act as a siderophore for certain microorganisms^{22.}

Although knowledge for treating the aforesaid diseases is advancing, there is at present no effective treatment against sepsis, acute respiratory distress syndrome (ARDS), septic shock, cytokine shock and systemic inflammatory responses, or macrophage activation syndrome (MAS) also called hemophagocytic lymphohistiocytosis (HLH), whether or not these diseases are caused by a viral infection, such as for example a coronavirus infection such as COVID-19. The COVID-19 disease linked with SARS-COV-2 particularly reflects the physiopathology of sepsis and illustrates the necessity of implementing blood purification therapies.

### TECHNICAL PROBLEM

Thus there is at present a need for development of novel means making it possible to treat the aforesaid diseases, said means in particular enabling the extraction of circulating molecules from the blood of a mammal and not exhibiting the disadvantages of the known systems, namely non-specificity of the circulating molecules to be extracted.

### SUMMARY

According to a first aspect, there is proposed a dialysis system capable of being connected to an extracorporeal blood circulation device comprising:
(i) a porous dialysis membrane, and
(ii) a vessel containing a dialysis fluid,
   characterized in that the dialysis fluid contains at least one additive selected from nanoparticles, polymers and biomolecules, said additive having a size greater than the cutoff threshold of said porous dialysis membrane, and contains or is constituted of at least one ligand specific to a circulating molecule of the blood of a mammal.

The characteristics disclosed in the following paragraphs can, optionally, be implemented in the dialysis system according to the invention. They can be implemented independently from one another or in combination with one another.

According to embodiment 1, the dialysis fluid contains at least one additive comprising or constituted of a ligand specific to the circulating molecules involved in the immune response mechanisms, preferably endotoxins, and/or immune response activator molecules, and/or pro-inflammatory cytokines, preferably IL6, IFN-γ and TNF-α.

According to embodiment 2, at least one of said ligands is an antibody, in particular a recombinant monoclonal antibody, a mixture of antibodies, an antibody fragment binding the antigen, a structural protein or a fusion protein, or else nanoparticles.

According to embodiment 3, at least one of said ligands is selected from medicines directed against interleukin-6 (Sirukumab, Olokizumab), interferon γ (Emapalumab), TNF-alpha (Etanercept, Infliximab, Adalimumab, Golimumab, Certolizumab), CCL2 (NOX-E36), interleukin-18 (Canakinumab), and mixtures thereof.

According to embodiment 4, the porous dialysis membrane has an area of at least 0.1 m².

According to embodiment 5, the additive is selected from nanoparticles having a mean diameter lying between 3 and 50 nm, for example nanoparticles of polysiloxane.

According to embodiment 6, the additive is selected from polymers, for example biocompatible polymers such as polysaccharides, or a biomolecule, and said additive has a size greater than 100 kDa, and preferably less than 1200 kDa.

According to embodiment 7, at least one of the ligands is specific to the circulating molecules involved in oxidative stress, in particular is a ligand specific to metal cations circulating in the blood.

According to embodiment 8, at least one of the ligands is a molecule complexing metal cations selected from the following complexing molecules: DOTA, DTPA, EDTA, TTHA, EGTA, BAPTA, NOTA, DOTAGA, DFO, DOTAM, NOTAM, DOTP, NOTP, TETA, TETAM, TETP and DTPABA, derivatives thereof and/or mixtures thereof. Preferably, said complexing molecule is capable of complexing trace metals selected from Cu, Fe, Zn, Mn, Co, Mg, and Ca, preferably Cu, Fe and Zn.

According to embodiment 9, the dialysis system comprises one or more additives selected from:
polysaccharides having a mass of at least 100 kDa and less than 2000 kDa, for example at a concentration lying between 0.1 and 10 g/l in the dialysis fluid, if applicable, onto which are covalently grafted molecules complexing metal cations, for example in a numerical proportion lying between 50 and 5000, and preferably between 100 and 1000 per polysaccharide,
ligands specific to circulating molecules of the blood and having a size greater than 100 kDa, preferably in a proportion lying between 1 μg/I and 1 g/l in the dialysis fluid, and/or
polysaccharides onto which ligands specific to the circulating molecules of the blood having a size lying between 10 and 100 kDa are covalently grafted, for example in a numerical proportion lying between 0.1 and 100 and preferably between 1 and 10 per polysaccharide.

Also proposed is a dialysis system as defined in the previous embodiments for use in preventing and/or treating a disease selected from sepsis, acute respiratory distress syndrome (ARDS), cytokine storm, septic shock and/or any other forms of systemic inflammatory response, macrophage activation syndrome (MAS) or hemophagocytic lymphohistiocytosis (HLH). Preferably, the dialysis system is used for treating a sepsis or cytokine storm caused by a bacterial, parasitic, fungal or viral infection, preferably a viral infection, for example coronaviruses with human respiratory tract tropism, in particular COVID-19.

Also proposed according to another aspect is use of an additive for dialysis fluid in a dialysis system according to the previous embodiments, said additive being selected from nanoparticles, polymers or biomolecules, and in that said additive comprises or is constituted of a ligand specific to a circulating molecule of the blood. Preferably, the ligand is specific to a circulating molecule involved in the mechanisms of an immune response, for example endotoxins, and/or immune response activator molecules, and/or pro-inflammatory cytokines, for example IL-6, IFN-γ and TNF-α.

According to a preferred embodiment of this aspect, said ligand is an antibody, in particular a recombinant monoclonal antibody, a mixture of antibodies, or else an antibody fragment binding the antigen.

According to a preferred embodiment of this aspect, the additive is selected from nanoparticles or polymers comprising a ligand specific to a molecule complexing metal cations circulating in the blood, in particular a trace metal selected from Cu, Fe, Zn, Mn, Co, Mg, and Ca, preferably Cu, Fe and Zn.

According to a preferred embodiment of this aspect, the additive has a size lying between 100 and 1200 kDa.

Also proposed is a dialysis fluid, for use in a dialysis system according to the previous embodiments, characterized in that it comprises an additive as previously defined, in an effective quantity of 10 to 100 nanomoles, preferably for a total volume of dialysis fluid lying between 0.5 and 10 liters.

According to a preferred embodiment, the dialysis fluid is used for the ex vivo capture of circulating molecules from the blood.

According to a preferred embodiment, the dialysis fluid is used for treating sepsis, cytokine release syndrome and/or any other form of systemic inflammatory response or cytokine storm.

According to a preferred embodiment, the dialysis fluid is used in order to limit the growth of a pathogen.

### DESCRIPTION OF DIAGRAMS

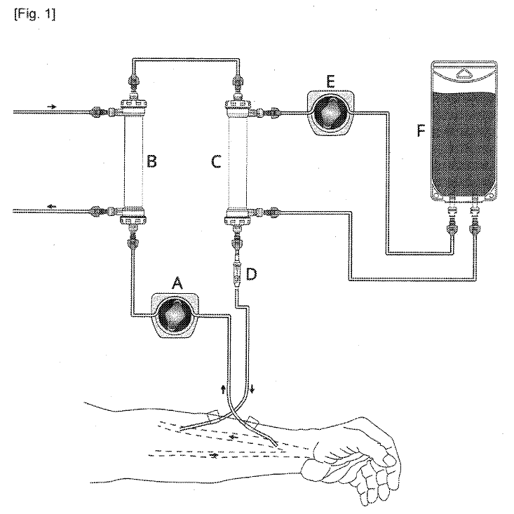

FIG. 1 shows a dialysis system connected to an extracorporeal circulation device according to a first embodiment. A: peristaltic pump; B: classical dialyzer; C: dialyzer with porous membrane according to the invention; D: bubble trap; E: peristaltic pump; F: dialysis fluid vessel.

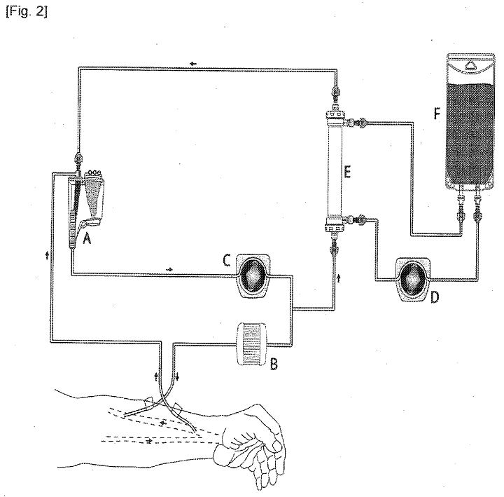

FIG. 2 shows a dialysis system connected to an extracorporeal circulation device according to a second embodiment. A: vessel; B: oxygenator; C and D: peristaltic pumps; E: dialyzer with porous membrane; F: dialysis fluid vessel.

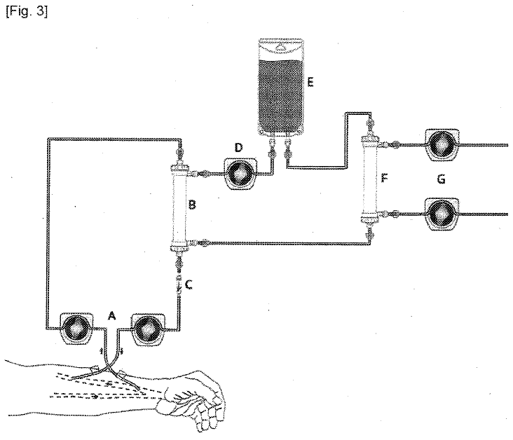

FIG. 3 shows a dialysis system connected to an extracorporeal circulation device according to a third embodiment. A: peristaltic pump; B: dialyzer with porous membrane; C: bubble trap; D: peristaltic pump; E: dialysis fluid vessel; F: classical dialyzer; G: dialysate circuit peristaltic pumps.

### DETAILED DESCRIPTION

The inventors have developed a dialysis system capable of being connected to an extracorporeal blood circulation device, said dialysis system enabling the extraction of circulating molecules from the blood of a mammal, and specifically.

Dialysis consists in placing the blood in contact with a sterile liquid (the dialysate) the composition whereof is close to that of the plasma (the liquid which makes up about 60% of the blood) across a membrane which serves as a filter. In the context of the present invention, this relates to systems of dialysis via an extracorporeal blood circulation.

In the sense of the present invention, “dialysis system” is understood to mean, the devices enabling purification of the blood (hemodialysis, hemofiltration or hemodiafiltration). In general they comprise a dialyzer, comprising an artificial, synthetic membrane, and a vessel containing a dialysate or dialysis fluid.

By way of example, suitable dialysis systems according to the invention are in particular presented in FIGS. 1 to 3. In these diagrams, the dialysis systems are connected to extracorporeal circulation devices, such as for example a dialyzer or a blood oxygenation circuit made up of a vessel and an oxygenator.

“Extracorporeal blood circulation device” is understood to mean a device enabling diversion of the venous blood flow into a circuit situated outside the body with a circulation flow rate of at least 10 ml/min. For example, the conventional hemodialysis systems or blood oxygenation devices.

“Circulating molecules of the blood” is understood to mean all the molecules in circulation in the blood, where said molecules may exist independently in free form or in aggregated or complexed form. By way of example and non-exhaustively, the circulating molecule may be a peptide, a protein, and in particular a glycoprotein, an immunoglobin, a cytokine, a metal cation, metal complexes or else the molecules known to the skilled person involved in the immune response mechanisms or else pro-inflammatory immune response activator molecules.

The dialysis system enables the extraction of circulating molecules in the blood the size whereof is less than the cutoff threshold of the dialysis membrane, and in particular circulating molecules the size whereof is less than 100 kDa, 50 kDa which constitutes an important threshold for the principal protein of human blood plasma, albumin (65 kDa), or even of the order of 20 to 30 kDa which is the threshold currently used for hemo(dia)filtration treatments in renal replacement therapy (RRT).

“Ligand” is understood to mean a molecule or part of molecule which binds specifically, preferably reversibly, to the circulating molecule. Advantageously, the specific ligand-circulating molecule bond is created owing to forces between molecules, such as ionic bonds, hydrogen bonds, hydrophobic interactions and Van der Waals forces or even the entropy variations linked with the release of solvation molecules during the close association between two complementary molecules. Thus, the ligand-circulating molecule interaction is reversible and stronger or weaker depending on the number and the nature of the bonds formed. It can moreover be very specific. The force of this interaction is defined by the affinity for the circulating molecule, and can for example be linked to the dissociation constant. Preferred examples of ligands include antibodies, artificial protein ligands, recombinant peptides or proteins (e.g. decoy receptors), or else molecules complexing metal cations.

“Antibodies” is understood to mean molecules of immunoglobulins and fragments thereof binding specifically to an antigen (a circulating molecule in the context of the present invention). The term “antibodies” in the sense of the present invention thus includes the antibodies and fragments thereof, as well as functional variants. The term “antibodies” also includes bispecific or multispecific antibodies. Natural antibodies are immunoglobulins formed of 4 polypeptide chains, two heavy H and two light L, capable of specifically binding an antigen, also referred to as circulating molecule in the context of the present invention. There are 5 types of antibody, IgG, IgM, IgD, IgA and IgE. The light chain generally includes 2 domains, a variable domain (VL) and a constant domain (CL). The heavy chain includes a variable domain (VH) and 3 constant domains (CH1, CH2, and CH3). The variable regions of the heavy chain and the light chain determine the antigen recognition specificity.

The VH and VL regions further contain hypervariable regions, the CDR regions designated respectively H-CDR1, H-CDR2 and H-CDR3 for the VH region, and L-CDR1, L-CDR2, and L-CDR3 for the VL region. An antibody can be characterized by the polypeptide sequence of its 6 CDRs or of the VH and VL regions.

In the sense of the invention, an antibody is specific to a circulating molecule, if it can bind an epitope of that circulating molecule. For example, it is capable of binding a epitope of a circulating molecule with a KD of 100 nM or less, 10 nM or less, 1 nM or less, 100 pM or less, or 10 pM or less.

The affinity constant of an antibody (or KD) can be measured in vitro by means of methods well known to the skilled person, in particular the surface plasmon resonance (SPR) methods of the Biacore® type (see for example, Rich RL, Day YS, Morton TA, Myszka DG. High-resolution and high throughput protocols for measuring drug/human serum albumin interactions using BIACORE®. Anal Biochem. 2001 Sep 15;296(2):197-207).

The term “antibodies” includes in particular the monoclonal antibodies, that is to say an antibody preparation of unique composition, which in particular exhibits a unique specificity and affinity for a particular epitope.

The term “antibodies” also includes non-natural antibodies modified, for example by mutation, humanization, or deletion of regions non-essential to the binding to its antigen, and fusion proteins comprising antibody fragments binding the antigen. Finally, the term “antibodies” includes antibodies chemically modified, in particular, in order to increase their molecular weights, for example by pegylation.

“Artificial protein ligand” (“scaffold protein” or “engineered protein” in English) is understood to mean a compound or protein fragments selected for their affinity towards specific circulating molecules. They are generally lighter than the antibodies, often easier to produce uniformly and chemically stable. Advantageously, the artificial protein ligands are of less than 50 kDa, preferably less than 30 kDa, and more preferably between 10 and 20 kDa. Such ligands present a good surface area. These artificial protein ligands can be selected from: ABD, Adhiron, Adnectin, Affibody, Affilin, Affimer, Affitin, Alphabody, Anticalin, Armadillo repeat proteins, Atrimer/tetranectin, Avimer/Maxibody, Centyrin and DARPin1.

Prevention and/or treatment is understood to mean a method aiming to reduce, block advancement of, hinder or eliminate one or more symptoms in an individual suffering from a pathology or disease causing this or these symptom(s) or capable of causing them. For example, in the case of an inflammatory disease, the treatment can consist in reducing, decreasing, blocking progression or hindering an excessive inflammatory reaction.

A first aspect of the invention relates to a dialysis system capable of being connected to an extracorporeal blood circulation device, said system comprising:
a porous dialysis membrane, and
a vessel containing a dialysis fluid,
   characterized in that the dialysis fluid contains an additive selected from nanoparticles, polymers and biomolecules, said additive having a size greater than the cutoff threshold of said porous dialysis membrane, and contains or is constituted of at least one ligand specific to a circulating molecule of the blood of a mammal.

By a particular choice of additive and/or ligand, the dialysis system according to the invention enables specific extraction of circulating molecules from the blood.

According to a particular embodiment, the blood of a mammal is a human blood.

According to a particular embodiment, the additive is diluted in the dialysis fluid. In other words, the additive is not immobilized on a fixed solid support. The additive remains in the dialysis compartment by reason of its size which is greater than the cutoff threshold of the dialysis membrane.

Dialysis systems are devices known to the skilled person and comprise a porous dialysis membrane as well as a vessel containing a dialysis fluid. The vessel can be an external vessel generally necessitating an additional device to circulate the dialysis fluid. This vessel can be directly included in the dialysis cartridge, the cartridge then constituting its own vessel.

The dialysis system according to the invention is capable of being connected to an extracorporeal blood circulation device. Such dialysis fluid vessel devices are for example described in the publication: Extrarenal purification in intensive care (Didier Journois, Frédéruqe Schorgen, 2003, Masson) or for example developed or marketed by the companies Baxter, Fresenius, Dialife, Asahi Kasei, Debiotech, Medtronic, Nipro, Torray and Braun.

According to another particular embodiment, the porous membrane of the dialysis system is advantageously a semi-permeable membrane.

According to another particular embodiment, the porous membrane has an exchange area of at least 0.1 m². Advantageously, the porous membrane has an exchange area lying between 0.1 and 4 m², preferably from 0.1 to 3 m² and more preferably from 0.5 to 2.5 m².

In the dialysis system according to the invention, the dialysis fluid contains an additive selected from nanoparticles, polymers and biomolecules, said additive having a size greater than the cutoff threshold of said porous dialysis membrane, and contains or is constituted of at least one ligand specific to a circulating molecule of the blood of a mammal.

“Cutoff threshold” is understood to mean the critical molar mass for which at least 90% of the solutes are retained by the membrane during a conventional treatment. Thus, in a preferred embodiment, the additive has a critical size not allowing it to cross the dialysis membrane. Consequently, the additive or a proportion at least greater than 90% of that additive remains within the dialysis fluid of only one side of the dialysis membrane. And preferably, the cutoff threshold is selected so that the additive is more than 92%, 94%, 96%, 98%, 99%, or even more than 99.9% retained in the dialysis fluid.

According to a particular embodiment, the additive has a size greater than 30, 40, 50, 60, 70, 80, 90 or 100 kDa. Preferably, the additive has a size greater than 100 kDa and less than 3000 kDa, more preferably greater than 100 kDa and less than 2000 kDa, and most preferably greater than 100 kDa and less than 1200 kDa.

In one embodiment, which can be combined with the previous embodiment, the dialysis membrane has a cutoff threshold less than or equal to 30, 40, 50, 60, 70, 80, 90 or 100 kDa. Preferably, the cutoff threshold lies between 5 and 100 kDa, more preferably from 5 to 50 kDa, more preferably from 10 to 30 kDa, and most particularly from 10 to 20 kDa.

According to a particular embodiment, the difference between the size of the additive the size of the cutoff threshold is at least 50 kDa and preferably greater than 100 kDa.

The size of the additive can also be defined by its mean diameter, in particular in the case of essentially spherical or globular structures, for example nanoparticles. The term “mean diameter” is then understood to mean the harmonic mean of the diameters of the additive, in particular the nanoparticles, the polymer or the biomolecule comprising or being constituted of a ligand specific to a circulating molecule of the blood.

The size distribution of nanoparticles or polymer can for example be measured by means of a commercial granulometer, such as a Malvern Zêta Sizer Nano-S granulometer based on PCS (English acronym for “Photon Correlation Spectroscopy”) which is characterized by a mean hydrodynamic diameter. A method for measurement of this parameter is also described in the standard ISO 13321:1996.

According to a first modification of the invention, the additive comprises one or more ligands specific to a circulating molecule of the blood. According to that modification, the ligand or ligands are directly or indirectly bound or grafted by covalent bonding onto their vector, for example a nanoparticle or a polymer, typically a vector the size whereof is greater than the cutoff threshold of the dialysis membrane. The vector can advantageously, in particular, make it possible to increase the size of the ligand or ligands in order to hinder their passage through the dialysis membrane. Several identical ligands can be covalently grafted onto one single vector (polymer or nanoparticles). Alternatively, different ligands, and in particular specifically binding different molecules can be covalently grafted onto one single vector.

The indirect bonding or the indirect grafting can be effected by a linker or molecular spacer between the additive and the ligand, said linker or ligand being covalently bound or grafted to the vector and to the ligand.

According to a second modification of the invention, the additive is constituted of the ligand specific to a circulating molecule of the blood. This signifies that the additive is the ligand as such.

The choice of the first or the second modification according to the invention depends in particular on the size of the specific ligand. In fact, certain ligands specific to the circulating molecules of the blood intrinsically have a size less than the cutoff threshold of the dialysis membrane and consequently must be directly or indirectly bound to a vector in order to remain in the dialysis fluid and not to pass through the dialysis membrane.

Conversely, in the case where the ligand intrinsically has a size greater than the cutoff threshold of the membrane, it cannot be bound to a nanoparticle or a polymer, and in that case it is considered as the additive as such.

Thus, depending on the circulating molecules to be extracted from the blood with the dialysis system according to the invention, the skilled person is able to determine with regard to the cutoff threshold of the dialysis membrane whether the additive utilized comprises a vector or is essentially constituted of said ligand specific to said circulating molecules.

According to a preferred embodiment, the additive is in solution or in suspension in an aqueous fluid, thus constituting a solution for dialysis fluid. According to this embodiment, the solution for dialysis fluid contains more than 0.01 mass % of additive, in particular more 0.1%, 0.2%, 0.3%, 0.4%, 0.5%, and preferably more than 0.5 mass % of additive. Advantageously, the solution for dialysis fluid contains from 0.1% to 5 mass % of additive, preferably from 0.1 to 3%, more preferably from 0.1 to 1%, and most particularly from 0.1 to 0.5 mass % of additive.

According to a preferred embodiment, the dialysis system according to the invention contains a dialysis vessel of capacity lying between 0.5 and 10 liters, preferably from 1 to 7 liters and more preferably from 1 to 5 liters. Preferably, in this embodiment, the dialysis fluid is recirculated within said dialysis system.

### Description of the Ligands According to the Invention

Suitable ligands according to the invention are such as described below, alone or in mixtures, and can advantageously be in solution.

According to a first modification at least one ligand is specific to a circulating molecule of the blood involved in the immune response mechanisms and/or of an immune response activator molecule.

According to a particular embodiment of this first modification, the ligand is specific to a pro-inflammatory cytokine or to a chemokine. Non-limiting examples of pro-inflammatory cytokines are in particular IFN-α, IFN-γ, IL-1β, IL-6, IL-12, IL-18, IL-33, TNF-α, and TGFβ. Non-limiting examples of chemokines are in particular chemokine ligand 2 (CCL2), chemokine ligand 3 (CCL3), chemokine ligand 5 (CCL5), interleukin-8 (CXCL8), chemokine ligand 9 (CXCL9) and chemokine ligand 10 (CXCL10).

According to a particular embodiment, the ligand or ligand mixture is specific to IL-6, IFN-γ, TNF-α, CCL2, CCL5, CXCL8, and/or CXCL10.

According to another particular embodiment, the ligand or ligand mixture is specific to a pro-inflammatory cytokine selected from IFN-α, IFN-γ, IL-12, IL-18, IL-33, and TGFβ, and/or to a chemokine selected from CCL2, CCL5, CXCL8, and CXCL10.

According to another particular embodiment of this first modification, the ligand specific to a circulating molecule of the blood is an antibody, in particular a recombinant monoclonal antibody, a mixture of antibodies, an antibody fragment binding the antigen, or a fusion protein containing an antibody fragment. Typically, these are antibodies or fusion proteins specifically directed against a pro-inflammatory cytokine or a chemokine, for example such as cited above and, preferably against interleukin-6, TNF-α, IFN-γ, CCL2 or IL-1β. Preferably, the antibody or antibody mixture is directed against a pro-inflammatory cytokine selected from IFN-α, IFN-γ, IL-12, IL-18, IL-33, and TGFβ, and/or a chemokine selected from CCL2, CCL5, CXCL8, and CXCL10.

According to another particular embodiment, the ligand is selected from medicines directed against a pro-inflammatory cytokine or a chemokine, preferably from medicines directed against interleukin-6, TNF-α, IFN-γ, CCL2, IL-1β, and mixtures thereof, more preferably against IFN-γand/or CCL2. In a more preferred embodiment, these medicines are medicines know to the skilled person, in particular for use in vivo, for example by subcutaneous or intravenous injection. Preferably, the ligand is selected from medicines directed against interleukin-6, IFN-γ and TNF-α, and more preferably from medicines directed against IFN-γ.

Examples of medicines directed against interleukin-6 are in particular Sirukumab, Siltuximab and Olokizumab.

Examples of medicines directed against TNF-alpha are in particular Etanercept, Infliximab, Adalimumab, Golimumab, and Certolizumab.

An example of a medicine directed against interferon gamma is in particular Emapalumab marketed under the name of Gamifant.

An example of a medicine directed against CCL2 is in particular NOX-36.

An example of a medicine directed against interleukin-1β is in particular Canakinumab.

According to another particular embodiment, the ligand is selected from Emapalumab and NOX-36.

According to a second modification which may if applicable be combined with the first modification, a ligand is specific to metal cations circulating in the blood, for example metal cations involved in oxidative stress.

According to a particular embodiment of this modification, the metal cations are preferably trace metals selected from the cations of the metals copper (Cu), iron (Fe), zinc (Zn), manganese (Mn), magnesium (Mg), cobalt (Co) and calcium (Ca), and most preferably Cu, Fe and Zn.

According to this embodiment, the ligand can be a molecule complexing metal cations.

Thus, the ligand can be selected from the following complexing molecules: DOTA (1,4,7,10-tetraazacyclododecane-N,N′,N″,N′″-tetraacetic acid), DTPA (diethylene triamine penta-acetic acid), EDTA (2,2′,2″,2′″-(ethane-1,2-diyldinitrilo)tetraacetic acid), TTHA (3,6,9,12-tetrakis(carboxymethyl)-3,6,9,12-tetrazatetradecane-1,14-dioic acid), EGTA (ethylene glycol-bis(2-aminoethylether)-N,N,N′,N′-tetraacetic acid), BAPTA (1,2-bis(o-aminophenoxy)ethane-N,N,N′,N′-tetraacetic acid), NOTA (1,4,7-triazacyclononane-1,4,7-triacetic acid), DOTAGA ((2-(4,7,10-tris(carboxymethyl)-1,4,7,10-tetraazacyclododecan-1-yl)pentanedioic acid), DFO (deferoxamine), amide derivatives such as for example DOTAM (1,4,7,10-tetrakis(carbamoylmethyl)-1,4,7,10-tetraazacyclododecane) or NOTAM (1,4,7-tetrakis(carbamoylmethyl)-1,4,7-triazacyclononane), as well as mixed carboxylic acid/amide derivatives, phosphonic derivatives such as for example DOTP (1,4,7,10-tetraazacyclododecane-1,4,7,10-tetrakis(methylene phosphonate)) or NOTP (1,4,7-tetrakis(methylene phosphonate)-1,4,7-triazacyclononane), cyclam derivatives such as TETA (1,4,8,11-tetraazacyclotetradecane-N,N′,N″,N′″-tetraacetic acid), TETAM (1,4,8,11-tetraazacyclotetradecane-N,N′,N″, N′″-tetrakis(carbamoylmethyl)), TETP (1,4,8,11-tetraazacyclotetradecane-N,N′,N″,N′″-tetrakis(methylene phosphonate)), siderophores or molecules derived from siderophores, derivatives thereof and/or mixtures thereof. Preferably, the ligand is a cyclic ligand such as DOTA or a derivative of DOTA, such as for example DOTAGA.

For good specificity with iron, the ligand can advantageously be selected from derivatives of siderophores, in particular deferoxamine (DFO).

Advantageously, the complexation constant log (KC1) of the complexing molecule for at least one metal cation is greater than or equal to 10, preferably greater than or equal to 15.

According to a particular embodiment, the complexation reaction can be a transmetallation, that is to say an exchange of two metal cations. In such a case, the ligand can be pre-complexed with a first metal cation which will subsequently be exchanged with the circulating metal cation circulant to be extracted from the blood. According to this embodiment, when the ligand is pre-complexed with a first metal cation the complexation constant log (KC1′) for the first metal cation is less than the complexation constant log (KC1) of the circulating metal cation to be extracted. Pre-complexation with Zn(II) or with an alkaline earth cation, for example Ca(II) or Mg(II), is particularly valuable, and makes it possible directly to supply a complement of trace elements which could be critical in the context of the patient's progression.

Advantageously, the dialysis system enables the extraction of metal cations from the blood of a mammal when the content of said metal cations is less than 1 ppm, preferably less than 0.1 ppm, more preferably less than 0.01 ppm.

Advantageously, the dialysis system enables the extraction of cytokines from mammalian blood when the content of said cytokines is less than 1 ppb, preferably less than 0.1 ppb, and still more preferably less than 0.01 ppb.

According to a third modification, the additive comprises a mixture of one or more ligands specific to circulating molecules of the blood involved in the immune response mechanisms and/or immune response activators as previously described (for example one or more antibodies directed against interleukin-6, TNF-α, IFN-γ, CCL2, and/or IL-1β) and of a ligand specific to metal cations circulating in the blood as previously defined.

According to a particular embodiment of this modification, the additive comprises one or more ligands specific to pro-inflammatory cytokines or to a chemokine, such as for example one or more medicines listed above, and one or more molecules complexing metal cations or trace metals, as previously defined. Preferably, the additive comprises one or more specific ligands such as an antibody or mixture of antibodies directed against a pro-inflammatory cytokine selected from IFN-α, IFN-γ, IL-12, IL-18, IL-33, and TGFβ, and/or a chemokine selected from CCL2, CCL5, CXCL8, and CXCL10, and one or more molecules complexing metal cations or trace metals such as DOTA or a derivative of DOTA, such as for example DOTAGA.

Advantageously, the ligand can be present in the dialysis fluid in a proportion lying between 1 picomolar and 1 nanomolar and/or between 1 μg/l and 1 mg/l.

### Description of the Additives According to the Invention

According to a first modification, the additive is essentially constituted of a specific ligand as previously defined and has a size greater than the cutoff threshold of the dialysis membrane.

According to a particular modification, the additive has a size greater than 50 kDa relative to the cutoff threshold of the membrane, and more preferably greater than 100 kDa relative to the cutoff threshold of the membrane.

According to a second modification, the additive comprises nanoparticles as vector.

According to this modification, the additive contains at least one ligand covalently bound or grafted onto the nanoparticle, where said ligand can be as previously defined.

The ligand can advantageously be grafted onto the nanoparticles in a mass proportion lying between 1 and 90%, preferably between 10 and 80%, and more preferably between 20 and 60%.

The nanoparticles preferably used according to this embodiment, can be based on silica or polysiloxane, preferably based on polysiloxane.

The term “nanoparticles based on silica or polysiloxane” is understood to mean nanoparticles characterized by a mass percentage of silica or of polysiloxane of at least 8%.

According to this embodiment, the nanoparticles can have a mean diameter lying between 5 and 50 nm.

According to this embodiment, the nanoparticles can have a mean size greater than 20 kDa and less than 1200 kDa.

According to a third modification, the additive comprises a polymer as vector onto which one or more ligands are covalently grafted (hereinafter polymer-based additive).

According to this modification, the polymer-based additive contains at least one covalently bound or grafted ligand, where said ligand can be as previously defined.

The term “polymer” is understood to mean any macromolecule formed from the covalent catenation of a very large number of repeating units which derive from one or more monomers.

According to a preferred embodiment, the polymer is biocompatible.

The polymers preferably used according to this modification are for example selected from polysaccharides, polyacrylamides, polyamines, polyethylene glycols, polyvinyl alcohols, polycarboxylic compounds, and mixtures thereof. Preferably, the polymer is a polysaccharide, and more preferably chitosan.

Advantageously, the polymer-based additive has a hydrodynamic diameter lying between 1 nm and 1 μm and/or from 100 kDa to 2000 kDa.

The number of ligands bound or grafted onto the polymer varies depending on the size of said ligand. For example, if the ligand has a size less than 10 kDa, it can advantageously be grafted onto the polymer in a numerical proportion lying between 10 and 5000, preferably between 50 and 2500, and more preferably between 100 and 1000 per polymer.

Likewise for example, if the ligand has a size lying between 10 to 100 kDa, it can advantageously be grafted onto the polymer in a numerical proportion lying between 0.1 and 100, preferably between 1 and 50, and more preferably between 1 and 10 per polymer.

More advantageously, the polymer-based additive can be present in the dialysis fluid at a concentration lying between 0.1 and 10 g/l.

According to a fourth modification, the additive is a biomolecule, for example a protein, a peptide, or a polypeptide, a nucleic acid, or a polysaccharide.

In the sense of the invention, the term “biomolecule” preferably refers to a natural biological molecule or derivatives thereof, and fragments comprising a region specifically binding a circulating molecule of the blood. In particular, a biomolecule is an immunoglobulin, a fusion protein, a structural protein, for example a recombinant protein and modified versions thereof. It can for example be peptide, polypeptides or recombinant proteins. The biomolecules can be modified, in particular to increase their molecular mass. The techniques for increasing the molecular mass of a peptide, polypeptide or recombinant proteins are well known to the skilled person and include in particular pegylation, hesylation or other similar techniques.

According to a preferred embodiment, the biomolecule is essentially constituted of a ligand specific to a circulating molecule of the blood as previously defined.

According to a fifth modification, the dialysis fluid comprises a mixture of additives as defined in the previous modifications.

### Preferred Embodiments of the Additive

According to a preferred embodiment, the additive comprises a polymer of chitosan onto which is grafted as ligand a molecule complexing metal cations. Preferably, the complexing molecule is then DOTAGA and/or DFO and/or one of the derivatives thereof.

According to another preferred embodiment, the dialysis fluid of the system according to the invention comprises nanoparticles of polysiloxane onto which is grafted as ligand a molecule complexing metal cations. Preferably, the complexing molecule is DOTAGA.

According to a particular embodiment, the dialysis fluid contains a mixture of additives. According to this embodiment, at least additive is a specific ligand such as an antibody or mixture of antibodies directed against a pro-inflammatory cytokine selected from IFN-α, IFN-γ, IL-12, IL-18, IL-33, and TGFβ, and/or a chemokine selected from CCL2, CCL5, CXCL8, and CXCL10, and at least one additive is a molecule complexing metal cations or trace metals, preferably DOTA or a derivative of DOTA, in particular DOTAGA, said molecule being grafted onto a polymer or a nanoparticle as previously defined.

According to another particular embodiment, the dialysis fluid of the system according to the invention contains several additives selected from:
polysaccharides having a mass lying between 100 kDa and 1200 kDa, for example at a concentration lying between 0.1 and 10 g/l in the dialysis fluid, if applicable, onto which are covalently grafted molecules complexing metal cations, for example in a numerical proportion lying between 50 and 5000, and preferably between 100 and 1000 per polysaccharide,
ligands specific to circulating molecules of the blood as previously defined, said ligand having a size greater than 100 kDa, preferably in a proportion lying between 1 μg/l and 1 g/l in the dialysis fluid, and/or
polysaccharides onto which ligands specific to the circulating molecules of the blood as previously defined are covalently grafted, said ligands having a size lying between 10 and 100 kDa, for example in a numerical proportion lying between 0.1 and 100 and preferably between 1 and 10 per polysaccharide.

Another subject of the invention likewise relates to an additive solution for dialysis fluid or a dialysis fluid comprising one or more additives as previously defined comprising or being constituted of a ligand specific to a circulating molecule as previously defined.

In particular, the additive solution for dialysis fluid can be constituted of a stock solution comprising an effective quantity of additive with regard to its use in the dialysis device, after dilution in the dialysis fluid.

The dialysis fluid contains an effective quantity of said additive, if applicable with other conventional constituents of a dialysis fluid.

By judicious choice of additive and/or ligand, the dialysis system according to the invention enables extraction of circulating molecules from the blood of a mammal, and specifically, in particular for therapeutic purposes. Also, the invention likewise relates to said solution of additive or dialysis fluid for the whole of the uses of the dialysis system, described in the present description.

Many diseases are in fact linked with the production or overproduction of molecules circulating in the blood, for example pro-inflammatory cytokines or chemokines, accumulation whereof creates a disequilibrium which can be harmful to the mammal if it persists for too long.

Thus, the dialysis system according to the invention, through the specific extraction of circulating molecules from the blood produced or overproduced in the course of a disease enables treatment and/or prevention of said diseases.

One of the advantages of the present invention is to avoid administration of active compounds directly to the patient. It advantageously makes it possible to reposition compounds, medicines, known to the skilled person for their ability to capture circulating molecules in the blood, for example, inflammatory cytokines, with a view to their use in a dialysis system for the ex vivo (and no longer in vivo) capture of said undesirable circulating molecules, and this for therapeutic purposes, for example for treating ARDS.

Consequently, another subject of the invention relates to use of the dialysis system as previously described, for treating a human patient in acute failure and/or in intensive care, by ex vivo capture of molecules circulating in the blood.

As previously described, ARDS is a syndrome associated with COVID-19. Moreover, the cytokine storm is considered as on of the principal causes of ARDS and of multiple organ failure and plays an important part in the process of worsening of the disease. Finally, like MERS and SARS, persons suffering from COVID-19 have elevated levels of pro-inflammatory molecules.

Thus, another subject of the invention relates to use of the dialysis system as previously described in preventing and/or treating diseases selected from sepsis, acute respiratory distress syndrome (ARDS), cytokine storm, septic shock, systemic inflammatory responses, and macrophage activation syndrome, also called hemophagocytic lymphohistiocytosis (HLH).

According to this subject, the dialysis fluid of the dialysis system contains at least one additive selected from nanoparticles, polymers and biomolecules as previously defined, said additive having a size greater than the cutoff threshold of said porous dialysis membrane, and comprises or is constituted of a ligand specific to a circulating molecule of the blood involved in immune response mechanisms and/or an immune response activator molecule, preferably the ligand is specific to a pro-inflammatory cytokine or a chemokine.

The additive and/or the ligand are as previously defined.

In fact, these diseases are associated with substantial release of pro-inflammatory molecules, in particular cytokines, and/or chemokine which is harmful to the body. The extraction of these molecules by the dialysis system according to the invention thus makes it possible to prevent or to treat the mammal.

According to a particular embodiment, the dialysis system according to the invention can be used in preventing and/or treating diseases selected from sepsis, acute respiratory distress syndrome (ARDS), cytokine shock and systemic inflammatory responses caused by a viral infection, preferably a coronavirus infection, in particular a COVID-19 infection.

As previously stated, oxidative stress is a situation harmful to the body which causes biological damage. It occurs when the quantity of pro-oxidant molecules is greater than that of anti-oxidants. Pro-oxidant molecules are mainly constituted of reactive oxygen species and reactive nitrogen species. Under the conditions of sepsis, overproduction of ROS and of RNS is observed both in the blood circulation and in the damaged organs.

Thus, another subject of the invention relates to use of the dialysis system as previously described in preventing and/or treating oxidative stress.

According to this subject, the dialysis fluid of the dialysis device contains at least one additive selected from additives based on nanoparticles, polymers or biomolecules as previously defined, said additive having a size greater than the cutoff threshold of said porous dialysis membrane, and comprises or is constituted comprises an additive containing or constituted of a ligand specific to the circulating molecules involved in oxidative stress.

The additive and/or the ligand are as previously defined.

According to a particular embodiment, the dialysis fluid of the dialysis device contains an additive comprising or constituted of a ligand specific to metal cations, more preferably to trace metals selected from the cations of the metals copper (Cu), iron (Fe), zinc (Zn), manganese (Mn), cobalt (Co), magnesium (Mg), and calcium (Ca), and most preferably Cu, Fe and Zn.

According to this embodiment, the ligand can be a molecule complexing metal cations.

Another subject of the invention relates to use of the dialysis system as previously described for reducing the growth or proliferation of a pathogen, for example reducing the bacterial burden, in a mammal.

According to this subject, the dialysis fluid of the dialysis system contains at least one additive selected from additives based on nanoparticles, polymers and biomolecules as previously defined, said additive having a size greater than the cutoff threshold of said porous dialysis membrane, and contains or is constituted of at least one ligand is selected from molecules complexing the metal cations Cu, Fe, Zn, Mn, Mg, and Ca.

Another subject of the invention relates to a method for treating a disease selected from sepsis, acute respiratory distress syndrome (ARDS), cytokine storm, septic shock, macrophage activation syndrome (MAS) or hemophagocytic lymphohistiocytosis (HLH), and systemic inflammatory responses in a patient having need thereof, said method comprising operation of a dialysis system as previously defined in which the dialysis fluid contains at least one additive selected from nanoparticles, polymers and biomolecules, said additive having a size greater than the cutoff threshold of said porous dialysis membrane, and contains or is constituted of at least one ligand specific to a circulating molecule of the blood involved in the immune response mechanisms and/or of an immune response activator molecule, preferably the ligand is specific to a pro-inflammatory cytokine or to a chemokine.

In one embodiment, the treatment method comprises in particular, a stage of applying the dialysis system in a subject having need thereof by means of a catheter on a vein of said subject, a stage of dialysis by extracorporeal circulation of the blood of said subject with a volume of dialysis fluid lying between 0.1 and 10 liters and during a time sufficient to allow the extraction of the circulating molecules involved in the immune response mechanisms and/or an immune response activator molecule.

According to a particular embodiment, the dialysis fluid is circulated at a rate lying between 10 and 300 ml/min, in direct flow or in counterflow.

According to a particular embodiment, the circulation rate lies between 40 and 100 ml/min.

According to a preferred embodiment, the dialysis fluid is recirculated in the dialysis system. This recirculation makes it possible to optimize the extraction of the circulating molecules from the blood for a given quantity of additive present in the dialysis fluid. In this manner, the final volume of dialysis fluid consumed is reduced, as are the quantities of additive to be used.

According to a particular embodiment, the dialysis system is operated for a period lying between 2 and 200 h, preferably 4 and 48 h.

This treatment method is particularly suitable for treating subjects suffering from viral infection, in particular a viral infection caused by a coronavirus such as COVID-19, but also for subjects in acute failure and/or in intensive care.

The additive and/or the ligand are as previously defined.

Preferably, the ligand is specific to a pro-inflammatory molecule, and more preferably to a pro-inflammatory cytokine, such as for example interleukin-6, interferon-γ or TNF-α. More preferably, the specific ligand is an antibody or mixture of antibodies directed against a pro-inflammatory cytokine selected from IFN-α, IFN-γ, IL-12, IL-18, IL-33, and TGFβ, and/or a chemokine selected from CCL2, CCL5, CXCL8, and CXCL10.

Another subject of the invention relates to a method for reducing oxidative stress in a patient having need thereof, said method comprising operating a dialysis system as previously defined in which the dialysis fluid contains at least one additive selected from nanoparticles, polymers and biomolecules, said additive having a size greater than the cutoff threshold of said porous dialysis membrane, and contains or is constituted of at least one ligand specific to metal cations circulating in the blood.

The additive and/or the ligand are as previously defined.

Preferably, the ligand is selected from molecules complexing trace metals selected from the cations of the metals copper (Cu), iron (Fe), zinc (Zn), cobalt (Co), manganese (Mn), magnesium (Mg), and calcium (Ca), and most preferably Cu, Fe and Zn.

In one embodiment, the treatment method comprises in particular, a stage of applying the dialysis system in a subject having need thereof by means of a catheter on a vein of said subject, a stage of dialysis by extracorporeal circulation of the blood of said subject, with a volume of dialysis fluid lying between 1 and 7 liters and during a time sufficient to allow the extraction of metal cations circulating in the blood.

According to a particular embodiment, the dialysis fluid is circulated at a rate lying between 10 and 300 ml/min, in direct flow or in counterflow.

According to a particular embodiment, the circulation flow rate lies between 40 and 100 ml/min.

According to a preferred embodiment, the dialysis fluid is recirculated in the dialysis system.

According to a particular embodiment, the dialysis system is operated for a period lying between 2 and 20 h, preferably 2 to 15 h.

This treatment method is advantageously suitable for treating subjects suffering from a viral infection, in particular a viral infection caused by a coronavirus such as COVID-19, but also for subjects in acute failure and/or in intensive care.

Another subject of the invention also relates to a method for reducing, limiting or stopping the growth of a pathogen in a patient having need thereof, said method comprising operating a dialysis system as previously defined in which the dialysis fluid contains at least one additive selected from nanoparticles, polymers and biomolecules, said additive having a size greater than the cutoff threshold of said porous dialysis membrane, and comprises or is constituted of at least one ligand is selected from molecules complexing the metal cations Cu, Fe, Zn, Mn, Mg, and Ca and more particularly molecules complexing Fe(III) cations.

In one embodiment, the treatment process comprises in particular, a stage of application of the dialysis system in a subject having need thereof by means of a catheter on a vein of said subject, a stage of dialysis by extracorporeal circulation of the blood of said subject, with a volume of dialysis fluid lying between 0.5 and 10 liters and during a time sufficient to allow the extraction of metal cations of Cu, Fe, Zn, Mn, Mg, and Ca circulating in the blood.

According to a particular embodiment, the dialysis fluid is circulated at a rate lying between 10 to 300 ml/min, in direct flow or in counterflow.

According to a particular embodiment, the circulation flow rate lies between 40 and 100 ml/min.

According to a preferred embodiment, the dialysis fluid is recirculated in the dialysis system.

According to a particular embodiment, the dialysis system is operated for a period lying between 4 and 100 h, preferably 4 to 48 h.

The present invention can also be defined according to the different embodiments described hereinafter.

Embodiment 1: Dialysis system capable of being connected to an extracorporeal blood circulation system comprising:
(a) a porous dialysis membrane, and
(b) a vessel containing a dialysis fluid,
   characterized in that the dialysis fluid contains at least one additive selected from nanoparticles, polymers and biomolecules, said additive having a size greater than the cutoff threshold of said porous dialysis membrane, and contains or is constituted of at least one ligand specific to a circulating molecule of the blood of a mammal.

Dialysis system according to embodiment 1, characterized in that said dialysis fluid contains at least one additive comprising or constituted of a ligand specific to the circulating molecules involved in the immune response mechanisms, preferably endotoxins, and/or immune response activator molecules, and/or pro-inflammatory cytokines, preferably IL6, IFN-γ and TNF-α.

Dialysis system according to embodiment 1 or 2, characterized in that at least one of said ligands is an antibody, in particular a recombinant monoclonal antibody, a mixture of antibodies, an antibody fragment binding the antigen, a structural protein or a fusion protein, or else nanoparticles.

Dialysis system according to any one of embodiments 1 to 3, characterized in that at least one of said ligands is selected from medicines directed against interleukin-6 (Sirukumab, Olokizumab), interferon γ (Emapalumab), TNF-alpha (Etanercept, Infliximab, Adalimumab, Golimumab, Certolizumab), CCL2 (NOX-E36), interleukin-1β (Canakinumab), and mixtures thereof.

Dialysis system according to any of embodiments 1 to 4, characterized in that said porous dialysis membrane has an area of at least 0.1 m2 .

Dialysis system according to one embodiments 1 to 5, characterized in that the additive is selected from nanoparticles having a mean diameter between 3 and 50 nm, for example nanoparticles of polysiloxane.

Dialysis system according to one embodiments 1 to 5, characterized in that the additive is selected from polymers, for example biocompatible polymers such as polysaccharides, or a biomolecule, and said additive has a size greater than 100 kDa, and preferably less than 1200 kDa.

Dialysis system according to any one of embodiments 1 to 7, characterized in that at least one of said ligands is specific to the circulating molecules involved in oxidative stress, in particular is a ligand specific to metal cations circulating in the blood.

Dialysis system according to any one of embodiments 1 to 8, characterized in that at least one of said ligands is a molecule complexing metal cations selected from the following complexing molecules: DOTA, DTPA, EDTA, TTHA, EGTA, BAPTA, NOTA, DOTAGA, DFO, DOTAM, NOTAM, DOTP, NOTP, TETA, TETAM, TETP and DTPABA, derivatives thereof and/or mixtures thereof.

Dialysis system according to embodiment 9, characterized in that said complexing molecule is capable of complexing trace metals selected from Cu, Fe, Zn, Mn, Co, Mg, and Ca, preferably Cu, Fe and Zn.

Dialysis system according to any one of embodiments 1 to 10, characterized in that it comprises one or more additives selected from:
polysaccharides having a mass of at least 100 kDa and less than 2000 kDa, for example at a concentration lying between 0.1 and 10 g/l in the dialysis fluid, if applicable, onto which molecules complexing metal cations are covalently grafted, for example in a numerical proportion lying between 50 and 5000, and preferably between 100 and 1000 per polysaccharide,
ligands specific to circulating molecules of the blood and having a size greater than 100 kDa, preferably in a proportion lying between 1 μg/l and 1 g/l in the dialysis fluid, and/or
polysaccharides onto which ligands specific to the circulating molecules of the blood having a size lying between 10 and 100 kDa are covalently grafted, for example in a numerical proportion lying between 0.1 and 100 and preferably between 1 and 10 per polysaccharide.

Dialysis system any one of claims 1 to 11, for use in preventing and/or treating a disease selected from sepsis, acute respiratory distress syndrome (ARDS), cytokine storm, septic shock and/or any other forms of systemic inflammatory response, macrophage activation syndrome (MAS) or hemophagocytic lymphohistiocytosis (HLH).

Dialysis system according to embodiment 12, for treating sepsis or cytokine storm caused by a viral infection, preferably a coronavirus infection, in particular a COVID-19 infection.

Use of an additive for dialysis fluid in a dialysis system according to any one of embodiments 1 to 11, characterized in that the additive is selected from nanoparticles, polymers or biomolecules, and in that said additive comprises or is constituted of a ligand specific to a circulating molecule of the blood.

Use of an additive for dialysis fluid according to embodiment 15, characterized in that the ligand is specific to a circulating molecule involved in the mechanisms of an immune response, for example endotoxins, and/or immune response activator molecules, and/or pro-inflammatory cytokines, for example IL-6, IFN-γ and TNF-α.

Use of an additive for dialysis fluid according to embodiment 14 or 15, characterized in that said ligand is an antibody, in particular a recombinant monoclonal antibody, a mixture of antibodies, or else an antibody fragment binding the antigen.

Use of an additive for dialysis fluid according to one of embodiments 14 to 16, characterized in that the additive is selected from nanoparticles or polymers comprising a ligand specific to a molecule complexing metal cations circulating in the blood, in particular a trace metal selected from Cu, Fe, Zn, Mn, Co, Mg, and Ca, preferably Cu, Fe and Zn.

Use of an additive for dialysis fluid according to any one of embodiments 14 to 17, characterized in that the additive has a size lying between 100 and 1200 kDa.

Dialysis fluid, for use in a dialysis system according to any one of embodiments 1 to 13, characterized in that it comprises an additive as defined according to one of embodiments 14 to 18, in an effective quantity of 10 to 100 nanomoles, preferably for a total volume of dialysis fluid lying between 0.5 and 10 liters.

Use of an additive for dialysis fluid according to one of embodiments 14 to 18, or of a dialysis fluid according to embodiment 19, for the ex vivo capture of circulating molecules from the blood.

Use of an additive for dialysis fluid according to one of embodiments 14 to 18, or of a dialysis fluid according to embodiment 19, for treating sepsis, cytokine release syndrome and/or any other form of systemic inflammatory response or cytokine storm.

Use of an additive for dialysis fluid according to one of embodiments 14 to 18, or of a dialysis fluid according to embodiment 19, in order to limit the growth of a pathogen.

Use of an additive according to one of embodiments 14 to 18, or of a dialysis fluid according to embodiment 19, in order to reduce oxidative stress.

Use according to any one of embodiments 20 to 23, for treating a human patient in acute failure and/or in intensive care, by ex vivo capture of molecules circulating in the blood.

### EXAMPLES

### Example 1

An extracorporeal circulation is installed on a patient, a peristaltic pump enables circulation of the blood at a flow rate of 200 ml/min. The blood then passes through a dialysis system of the Theralite type, having 2.1 m² of membranes based on polyarylethersulfone and polyvinylpyrrolidone and a high cutoff threshold (HCO) allowing the passage of molecules with a molecular weight up to 45 kDa.

A vessel containing 3 liters of dialysis fluid is then connected to the dialysis cartridge and set in recirculation within the cartridge by a peristaltic pump. The rate of circulation of the dialysis fluid is set at 100 ml/min. The dialysis fluid is obtained by mixing 3 liters of conventional dialysis solution (for example PrismaSol or Prismocal) and 100 ml of a 10 g/l solution of chitosan onto which are grafted complexing molecules of DOTAGA (containing of the order of 1 millimole of complexant DOTAGA).

100 ml of a saline solution containing 1 g/l of Golimumab or equivalent (anti-TNF-α antibody) and 1 g/l of Olokizumab or equivalent (anti-IL-6 antibody) and 0.1 g/l of Emapalumab (anti IFN-γ antibody) are added to this solution.

The dialysis system is circulated for 12 hours. During these 12 hours, the treatment makes it possible to specifically extract metal cations, in particular of iron and copper, and pro-inflammatory cytokines: IL-6 and TNF-α from the blood.

### Example 2

A patient is connected to a dialysis system under an operating condition enabling continuous veno-venous hemodialysis (CVVHD). The dialysis monitor is a prismaflex system.

An HF20 kit (Prismaflex HF 20 set) is used, it contains 0.2 m² of PAES (Polyarylethersulfone) dialysis membranes.

100 ml of a MEX-CD1 solution containing 10 g/l of chitosan-DOTAGA and 10 ml of a 1 g/l solution of Sirikumab (anti-IL6 antibody) are successively introduced into a 5 L bag of PrismaSol (or Prismocal). This mixture is then used as dialysis fluid solution.

The dialysis fluid is then set in circulation at a rate of 1.25 L/hour in counterflow. The circulation flow rate of the blood is set at 60 ml/min.

After 4 h of treatment, the dialysis system makes it possible to specifically reduce the quantity of iron and of copper circulating in the blood, as well as the quantity of cytokine IL-6. Such a system thus makes it possible to treat a patient presenting septic shock.

To increase efficacy, the operation is repeated at least 4 times, using the same liquid after recirculation. After five uses, the dialysis cartridge is changed, as is the dialysis fluid, reconstituting this as initially performed.

10 ml of a 1 g/l solution of antibody of the Adalimumab type are added in order to specifically extract a part of the TNF-α molecules circulating in the blood and thus make it possible to limit immune runaway.

### LIST OF CITED DOCUMENTS

For all intents and purposes, the following non-patent items are cited:
P. Mehta, D. F. McAuley, M. Brown, E. Sanchez, R. S. Tattersall, J. J. Manson, COVID-19: consider cytokine storm syndromes and immunosuppression, The Lancet, 2020. 395, 1033-1034.
Q. Ye, B. Wang, J. Mao, Cytokine Storm in COVID-19 and Treatment, Journal of Infection, 2020. 10.1016/j.jinf.2020.03.037.
M. A. Wutjewicz, A. Dylczyk-Sommer, A. Aszkielowicz, S. Zdanowski, S. Piwowarczyk, R. Owczuk, COVID-19-what should anaethesiologists and intenisvists know about it, Anaesthesiol. Intensive Ther., 2020. 52, 34-41.
X. Li, M. geng, Y. Peng, L. Meng, S. Lu, Molecular immune pathogenesis and diagnosis of COVID-19, Journal of Pharmaceutical Analysis, 2020.
C. Huang, Y. Wang, X. Li, L. Ren, J. Zhao, Y. Hu, L. Zhang, G. Fan, J. Xu, X. Gu, Z. Cheng, T. Yu, J. Xia, Y. Wei, W. Wu, X. Xie, W. Yin, H. Li, M. Liu, Y. Xiao, H. Gao, L. Guo, J. Xie, G. Wang, R. Jiang, Z. Gao, Q. Jin, J. Wang, B. Cao, Clinical features of patients infected with 2019 novel coronavirus in Wuhan, China, 2020. 395, 497-506.
L. Li, R. Li, Z. Wu, X. yang, M. Zhao, J. Liu, D. Chen, Therapeutic strategies for critically ill patients with COVID-19, Ann. Intensive Care, 2020. 10, 45.
C. Zhang, Z. Wu, J.-W. Li, H. Zao, G-Q. Wang, The cytokine release syndrome (CRS) of severe COVID-19 and Interleukin-6 receptor (IL-6R) antagonist Tocilizumab may be the key to reduce the mortality, International Journal of Antimicrobial Agents, 2020.
H. Li, L. Liu, D. Zhang, J. Xu, H. Dai, N. Tang, X. Su, B. Cao, SARS-COV-2 and viral sepsis: observations and hypotheses, 2020.
Q. Ruan, K. Yang, W. Wang, L. Jiang, J. Song, Clinical predicators of mortality due to COVID-19 based on an analysis of data of 150 patients from Wuhan, China, Intensive Care Med., 2020.
B. M. Henry, M. H. Santos de Oliveira, S. benoit, M. Plebani, G. Lippi, Hematologic, biochemical and immune biomarker abnormalities associated with severe disease and mortality in coronavirus disease 2019 (COVID-19): a meta-analysis, Clin. Che. Lab. Med., 2020.
J. Ma, P. Xia, Y. Zhou, Z. Liu, X. Zhou, J. Wang, T. Li, X. Yan, L. Chen, S. Zhang, Y. Qin, X. Li, Potential effect of blood purification therapy in reducing cytokine storm as a late complication of critically ill CoVID-19 patients, Clin. Immunol., 2020. 214, 108408.
Y. Xia, N. Farah, A. Maxan, J. Zhou, C. Lehmann, Therapeutic iron restriction in sepsis, Medical Hypotheses, 2016, 89, 37-39.
B. G. Chousterman, F. K. Swirski, G. F. Weber, Cytokine storm and sepsis disease pathogenesis, Semmin. Immunopathol., 2017, 39, 517-528.
P. M. Honore, E. Hoste, Z. Molnar, R. Jacobs, O. Joannes-Boyau, M. L. N. G. Malbrain, L. G. Forni, Cytokine removal in human septic shock: where are we and where are we going, Ann. Intensive Care, 2019, 9, 56.
T. Rimmelé, J. A. Kellum, Clinical review: blood purification for sepsis, Critical Care, 2011, 15, 205.
S. Vera, R. Martinez, J. Guillermo Gormaz, A. Gajardo, F. galleguillos, R. Rodrigo, Novel relationships between oxidative stress and angiogenesis-related factors in sepsis: new biomarkers and therapies. Annals of Medicine, 2015, 47, 289-300.
S. Islam, S. Jarosch, J. Zhou, M. del Carmen Parquet, J. T. Toguri, P. Colp, B. E. Holbein, C. Lehmann, Anti-inflammatory and anti-bacterial effects of iron chelation in experimental sepsis, 2016, 200, 266-273.
F. Collin, Chemical basis of reactive oxygen species reactivity and involvement in neurodegenerative diseases, Int. J. Mol. Sci., 2019, 20, 2407.
P. Lan, K. -H. Pan, S. -J. Wang, Q. -c. Shi, Y. -X. Yu, Y. Fu, Y. Chen, Y. Jiang, X.T. -T. Hua, J. -C. Zhou, Y. -S. Yu, High serum iron level is associated with increased mortality in patients with sepsis, Sci. Rep., 2018, 8, 11072.
I. Akkas, N. Ince, M. A. Sungur, Serum trace element and heavy metal levels in patients with sepsis, Aging Male, 2020.
Y. Xia, N. Farah, A. Maxan, J. Zhou, C. Lehmann, Therapeutic iron restriction in sepsis, Medical Hypotheses, 2016, 89, 37-39.
S. Islam, M. Jobayer Chisti, M. Ahmed, N. Anwar, C. Lehmann, Bacterial resistance in pneumonia in developing countries—A role for iron chelation, Trop. Med. Infect. Dis., 2019, 4, 59.

1. A method for reducing oxidative stress in a patient having need thereof, said method comprising operating a dialysis system comprising
(a) a porous dialysis membrane, and
(b) a vessel containing a dialysis fluid, wherein the dialysis fluid contains an additive selected from polymers, said additive having a size greater than the cutoff threshold of said porous dialysis membrane, and contains or is constituted of at least one ligand specific to metal cations circulating in the blood,
wherein said patient is suffering from sepsis or viral infection.

2. The method according to claim 1, wherein the at least one ligand is present in the dialysis fluid in a proportion of 1 picomolar to 1 nanomolar and/or 1 μg/l to 1 mg/l.

3. The method according to claim 1, wherein said porous dialysis membrane has an area of at least 0.1m².

4. The method according to claim 1, wherein the at least one additive is nanoparticles having a mean diameter of 3 to 50 nm, said at least one ligand being covalently grafted onto said nanoparticles.

5. The method according to claim 1, wherein the at least one additive is a biocompatible polymer of a size greater than 100 kDa and less than 1200 kDa, and said at least one ligand is covalently grafted onto said polymer.

6. The method according to claim 1, wherein the at least one ligand specific to metal cations is a molecule that complexes metal cations and is selected from the group consisting of DOTA, DTPA, EDTA, TTHA, EGTA, BAPTA, NOTA, DOTAGA, DFO, DOTAM, NOTAM, DOTP, NOTP, TETA, TETAM, TETP, and DTPABA, derivatives thereof and mixtures thereof.

7. The method according to claim 6, wherein said molecule that complexes metal cations is capable of complexing trace metals selected from Cu, Fe, Zn, Mn, Co, Mg, and Ca.

8. The method according to claim 1, wherein said dialysis system is connected to an extracorporeal blood circulation device.

9. The method according to claim 8, comprising:
connecting the dialysis system to the subject by means of a catheter in a vein of said subject,
dialyzing blood of said subject by extracorporeal circulation, wherein the step of dialyzing is performed for a period of time sufficient to allow extraction of metal cations circulating in the blood.
